(19) United States
(12) Patent Application Publication
(10) Pub. No.: US 2019/0282300 A1
(43) Pub. Date: Sep. 19, 2019

(54) PROJECTED FLAP DESIGN

(71) Applicant: THE REGENTS OF THE UNIVERSITY OF CALIFORNIA, OAKLAND, CA (US)

(72) Inventors: Ross Sayadi, Orange, CA (US); Derek A Banyard, Orange, C (US); Mustafa Chopan, Gainesville, FL (US)

(73) Assignee: THE REGENTS OF THE UNIVERSITY OF CALIFORNIA, Oakland, CA (US)

(21) Appl. No.: 16/351,230

(22) Filed: Mar. 12, 2019

Related U.S. Application Data
(60) Provisional application No. 62/642,252, filed on Mar. 13, 2018.

(51) Int. Cl. A61B 34/10

(52) U.S. Cl. CPC A61B 34/10; A61B 17/322; A61B 2034/105

(57) ABSTRACT

A method of creating a skin flap includes selecting a skin flap configuration from a database of pre-existing skin flap configurations; and projecting the selected skin flap configuration onto an area of skin.

### CROSS REFERENCE TO RELATED APPLICATIONS

This application claims the benefit of and priority to U.S. provisional application No. 62/642,252 filed Mar. 13, 2018 which is incorporated herein by reference in its entirety.

### BACKGROUND OF THE INVENTION

The present invention generally relates to surgical skin flaps or markings and, more particularly, to apparatus and methods for creating surgical skin flaps.

Surgeons in all specialties utilize markings for planning of their operative incisions. Despite the vast amount of technological advancements seen in the operating room, surgical markings are still drawn free-hand as “best estimates.” Although there is an exhaustive literature devoted to their mathematics and biomechanics, reproducibility can prove challenging. Surgical markings drawn free-hand and to the surgeon's best estimate, lead to potential mistakes, increased procedure time and patient risk.

The prior art has not utilized a technology/imaging system to delineate important characteristics of the skin and anatomical area being reconstructed, and that feeds into an algorithm that selects the best surgical flap and area to place the reconstruction, and makes changes to the flap (incision length and angles) to personalize it to the patient's anatomical defect.

As can be seen, there is a need for improved apparatus and methods for creating surgical skin flaps.

### SUMMARY OF THE INVENTION

In one aspect of the present invention, a method of creating accurate skin flap comprises selecting a skin flap marking configuration from a database of pre-existing skin flap configurations; and projecting the selected skin flap configuration onto an area of skin requiring reconstruction.

In another aspect of the present invention, a system for creating a skin flap comprises a database of pre-existing skin flap configurations; a processor in communication with the database; and a projector in communication with the database and the processor; wherein the projector is configured to project a selected, pre-existing skin flap configuration onto an area of the skin.

In a further aspect of the present invention, a system for creating a skin flap comprises a database of pre-existing skin flap configurations; a processor in communication with the database; a projector in communication with the database and the processor; a thermal imaging camera in communication with the database and the processor.

These and other features, aspects and advantages of the present invention will become better understood with reference to the following drawings, description and claims.

### BRIEF DESCRIPTION OF THE DRAWINGS

FIG. 1A is a block diagram of a system for creating a skin flap according to an embodiment of the present invention.

FIG. 1B is a flow chart of a method for creating a skin flap according to an embodiment of the present invention.

FIG. 10 is a flow chart of a method for creating a skin flap according to another embodiment of the present invention.

FIG. 2 is a user interface of a system for creating a skin flap according to an embodiment of the present invention.

FIG. 3 is an image of a projector in a system for creating a skin flap according to an embodiment of the present invention.

FIG. 4 are images of comparative bilobed flaps according to an embodiment of the present invention versus hand drawn.

FIG. 5 is a table of comparative characteristics of bilobed flaps according to an embodiment of the present invention versus hand drawn.

FIG. 6 are images of comparative rhomboid flaps according to an embodiment of the present invention versus hand drawn.

FIG. 7 is a table of comparative characteristics of rhomboid flaps according to an embodiment of the present invention versus hand drawn.

FIG. 8 is a table of survey results from using the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, the present invention provides apparatus and methods of skin reconstructive surgery and, in particular, the creation of skin flaps or markings. In general, the present invention provides prefabricated but modifiable surgical markings, in a database, which can be projected onto a patient.

Optionally, the present invention can overlay surgical markings on the picture of the blood supply of a patient's defect, such as by the use of a FLIRT″ one pro camera. Pictures of the patient's blood supply may be stored in a database. A processor and memory of the present invention may analyze a photograph of the patient's defect and select the best reconstructive option from a database.

Optionally, the present invention can overlay a 3D picture of the reconstruction area, and the selected surgical marking can be overlaid on the 3D picture.

According to the present invention, a user can interface with the system via a projector. The surgical markings can be projected on to a patient prior to or during surgery. The projection can act as a stencil. In addition, from a database, the present invention enables the user to select an image which delineates the patient's blood supply, and the thermal image and/or 30 image, can be used as a background to the markings. The present invention can be used, for example, by general surgeons, plastic and reconstructive surgeons, dermatologists, otoloylngoloists, Mohs surgeons, oculoplastic surgeons, and oral maxillofacial surgeons. This present invention can also be used as a training tool for resident surgeons to help them perform surgical planning and improve the accuracy of their surgical markings.

In the present invention, the user application may run on the Android™ operating system, or other operating systems, and can be deployed on the majority of Android™ devices through an application download, such as an Android™ Package Kit (APK). Or the user application can be added to the Google Play Store where it can be downloaded (publicly or privately). The application can use Java as the primary language, with some minor XML (markup/formatting language) for the user interface. The user interface may include a splash screen with the author information that leads into the main page which provides basic information regarding the different flaps (images) available for projection. Once the user selects their desired flap, the image is displayed.

According to the present invention, depending on the flap specifications, options (e.g., angles, lengths, number of shapes, and flap orientation) can be available to customize what is required by tapping on the display screen to display a hidden menu. Further options can be available on the hidden menu, such as changing the background to include a custom image (e.g., photograph of a wound that requires reconstruction), which is also zoomable through the menu. The zoom ability ensures ease of use in order to reach the desired zoom on both the object and the background image. The selected flap can then be overlaid using the operating system on the background image and serves as a means of surgical planning.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable media having computer readable program code embodied thereon.

Any combination of one or more computer readable storage media may be utilized. A computer readable storage medium is an electronic, magnetic, optical, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. In the context of this document, a computer readable storage medium is any tangible medium that can store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable storage medium that can direct a computer, other programmable data processing apparatus, or other devices to function in a particular manner, such that the instructions stored in the computer readable storage medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

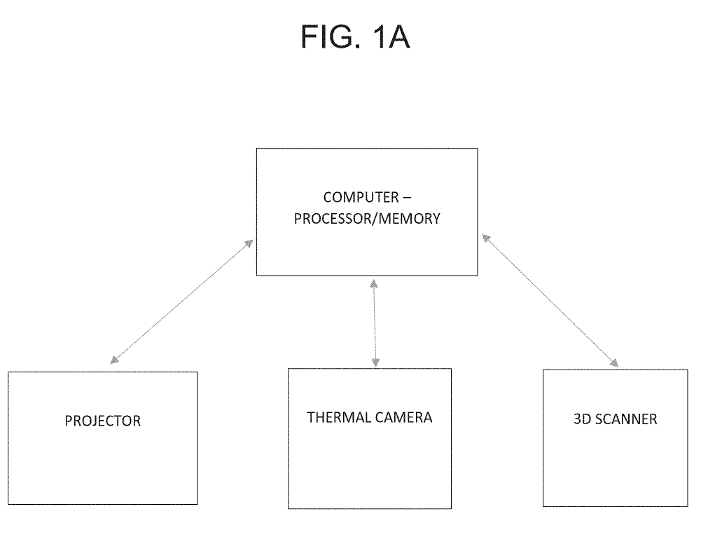

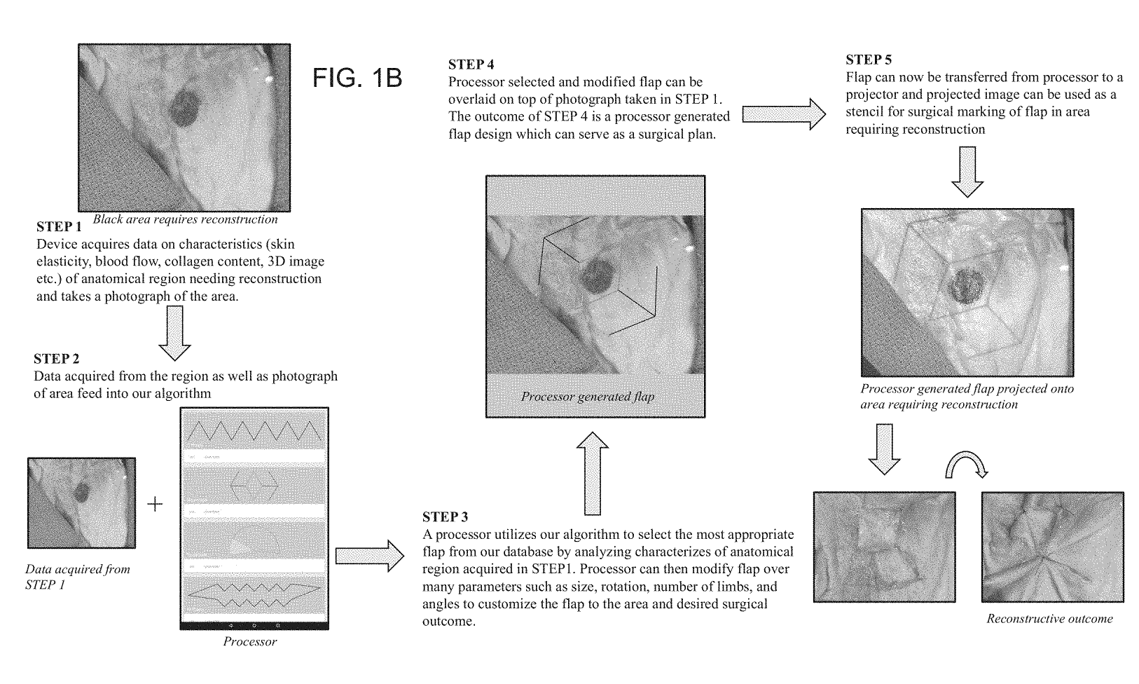

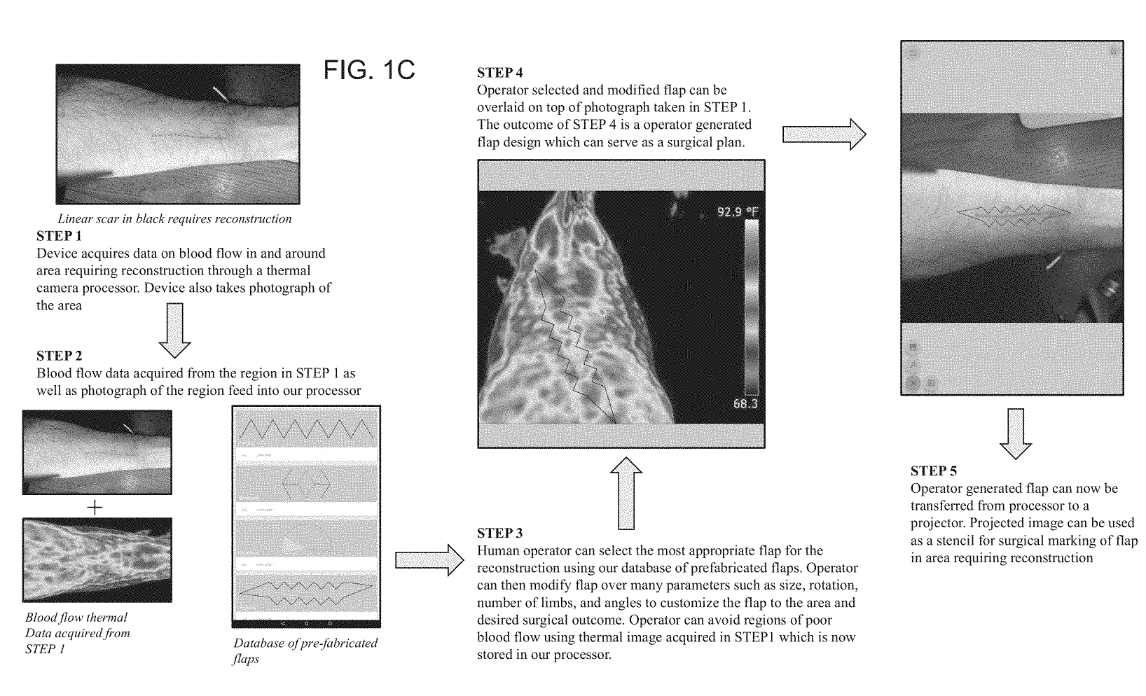

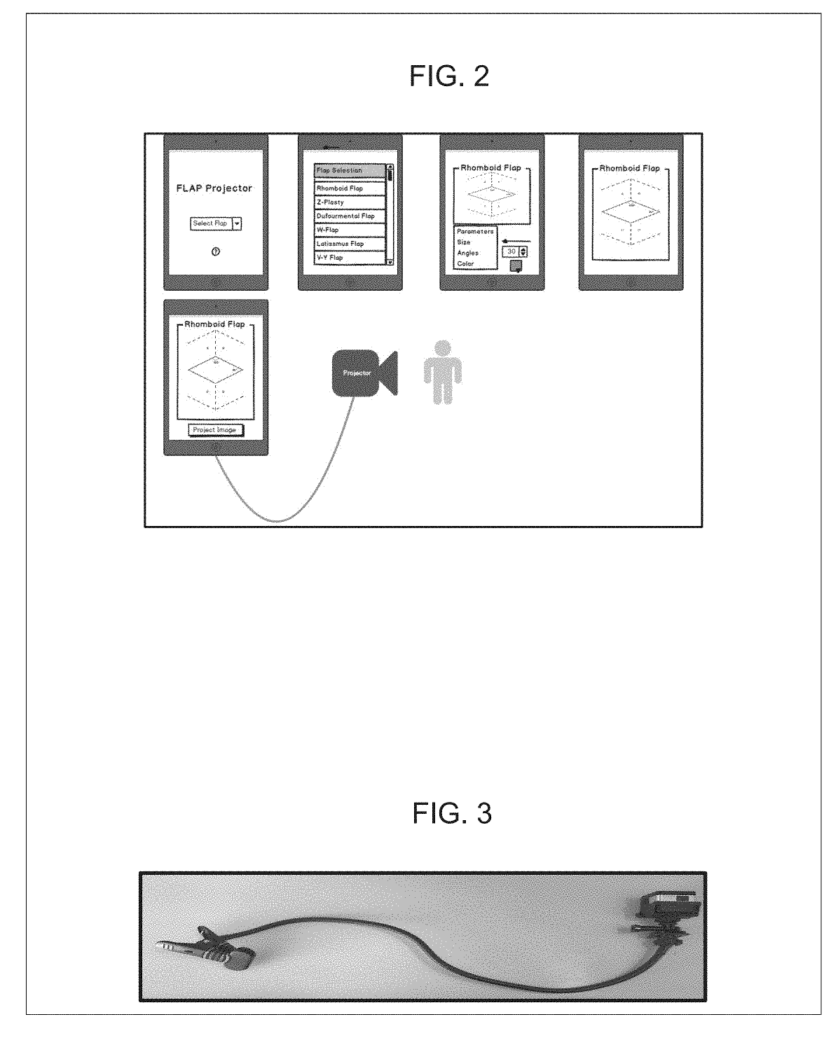

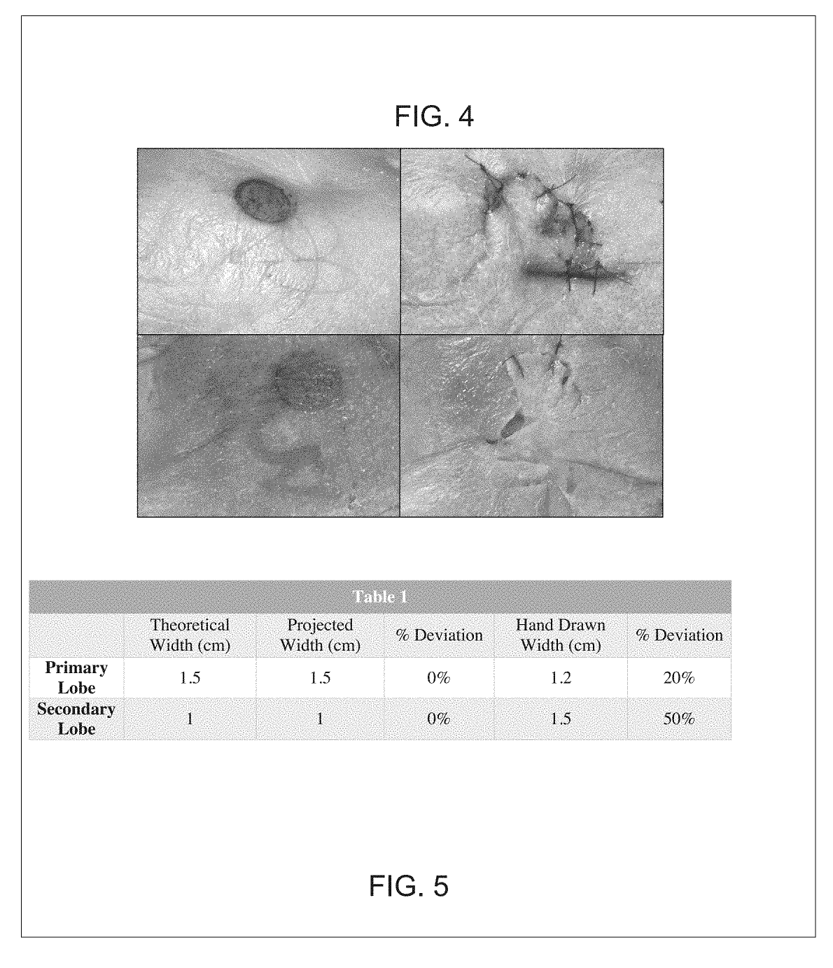

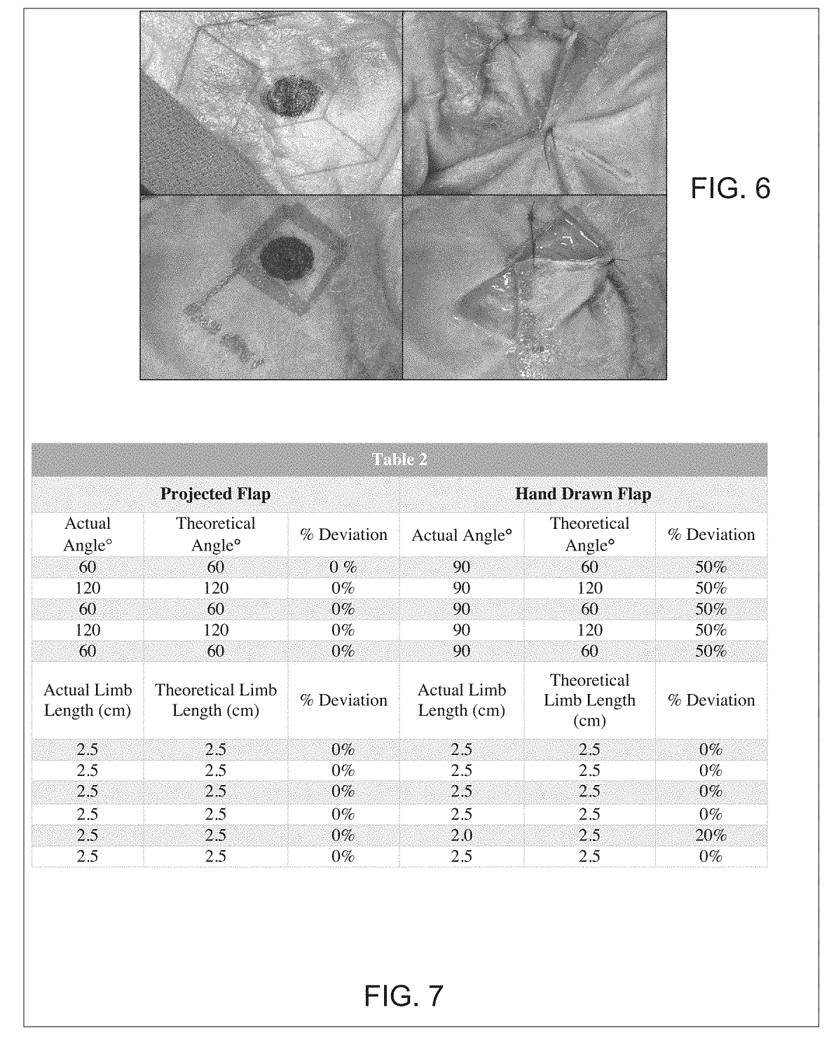

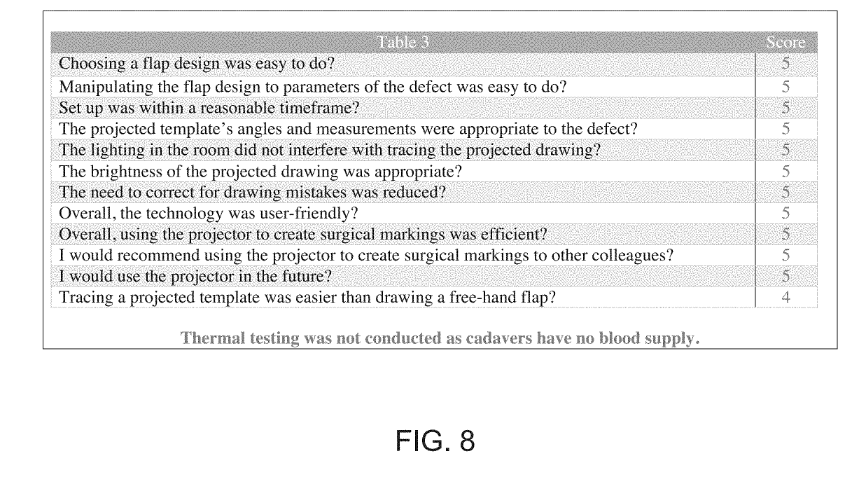

In FIG. 1A, a system for creating a surgical flap or marking may include a computer having a processor and memory. The computer may be in communication with a projector, thermal imaging camera (2D or 3D), and 3D scanner. The functions of the foregoing is described below.

FIGS. 1B-1C are flow charts of exemplary methods of the present invention. A thermal imaging camera may take a picture(s) of the patient's reconstructive area for which a skin flap is needed. The thermal imaging camera may be separate from a projector (FIG. 3) or part of an integrated thermal camera and projector. The thermal picture(s) may include an image of the patient's blood supply, and may then be stored in a database.

A 3D scanner may also be used to take images of the reconstruction area, such as skin surface topography and tissue volume. These images may be stored in a database.

Other imaging modalities may be used to analyze skin and determine, for example, color, texture, hair content, elasticity, collagen content, elastin content, and water content. These images may be stored in a database.

Based on the thermal picture(s) which can show areas of sufficient blood supply, an area of skin for a skin flap can be selected. Based on the foregoing and/or the characteristics of the reconstruction area obtained from other imaging modalities—such as skin surface topography, skin color, skin texture, hair content, skin elasticity, collagen content, defect size, and defect location—a skin flap may be selected from a database containing pre-selected skin flap configurations. The user may select the flap. Alternatively, a software program may select the flap, through artificial intelligence based on the data acquired from imaging modalities and using our algorithm to accomplish best aesthetic outcomes.

In the case of using a software program to select a flap, the program may, upon execution by a processor:
1. compare characteristics of the patient's areas requiring reconstruction to what is considered normal for his/her age, skin type etc.
2. select appropriate flap for the area from a database of flaps stored on our app and website. This selection process is made by the algorithm by combining information acquired by imaging systems and flap most commonly used for that defect type.
3. alter the geometry and position of the flap (1) (size of flap limbs, angles etc.) to better fit the area requiring reconstruction therefore personalizing the flap to the patient. This personalization is dependent on (1) location of defect (2) vascular nature of the territory (3) skin elasticity (4) skin color (5) skin texture (6) biological characteristics such as but not limited to collagen, elastin and water content (7) what the patient desires (i.e., breast size after breast reduction).
4. make corrections to selected flap when projected to account for topographic/3D/contour specific characteristics of the area.

Whether user selected or computer selected, the selected flap may then be modified or customized based on user selected modifications, such as by size, rotation, and angles.

An image of the selected flap, either modified or not, may then be projected (via the integrated thermal imaging camera/projector or separate projector) onto the reconstruction area of skin—with or without the thermal image projected onto such area. Similarly, the image of the selected flap may be projected onto the reconstruction area of skin with or without the 2D or 3D topography image projected onto such area. The same applies to any other imaging modality. Using at least the projected flap image, a skip flap can be traced onto the patient using a marking pen, for example. The tracing can then be used to create the skin flap.

In FIG. 2, an exemplary user interface of the system of the present invention is shown. A specific flap configuration can be selected from a database of pre-selected flap configurations. The selected configuration may then be modified and then projected onto the patient.

### Examples

Patient with dog bite to cheek over the zygomatic process comes in 4 weeks after injury for reconstruction of her defect. Reconstructive surgeon takes thermal, 3D and biochemical image using our device. Information obtained using these imaging techniques will be processed by our algorithm to select, modify/personalize and account for 3D projection of flap on the patient.

Woman comes in with severe ptosis of her breast and would like a breast lift (mastopexy). Plastic Surgeon uses our imaging system to acquire information on the patient's breast including vasculature, volume, degree of ptosis compared to what is normal or her age. Breast reduction flap will then be selected and modified based on these parameters using our algorithm. Physician will project personalized flap onto the patient and utilize it as a stencil.

Local flaps were designed based on their mathematical descriptions, as outlined in The Planning of Local Plastic Operations On the Body Surface: Theory and Practice by A. A. Limberg and other sources, all of which are incorporated herein by reference. Flap designs were projected onto the tissue with the Insignia Portable Pico Projector Cube (Insignia, Minneapolis, Minn.).

Cadaveric face specimens were used. As a method of standardization, mirroring defects (solid-colored circles) were created with respect to the midline for each specimen. The defects differed with respect to size and location unilaterally, but were consistently and accurately represented on the other half of the specimen. A hand-drawn flap and a projected reconstructive flap were then applied to each defect. The type of reconstructive local flap (i.e., rhomboid, bilobed) was consistent for each set of defects.

An experienced, board-certified plastic surgeon, who was blinded to the experiment, was first instructed to design a flap free-handed and reconstruct the defect. On the opposite side of the same specimen, the surgeon was instructed to utilize the projected flap in reconstruction. The free-hand and projected flaps were then analyzed and compared based on expected geometric parameters and overall result. Following the procedure, the surgeon completed a survey regarding projector use in local-flap based reconstruction.

In FIG. 4, the top row of images are of a projected bibbed flap according to the present invention. The bottom row of images are of a hand drawn bilobed flap.

In FIG. 5, a table of percent deviation in width of primary and second lobes of projected and hand drawn bilobed flaps is shown. The hand drawn flap had a 20% and a 50% deviation from a theoretical flap width respectively in the primary and secondary lobes when compared to published theoretical parameters. The projected flap had 0% deviation from the theoretical flap width in both lobes.

In FIG. 6, the top row of images are of a projected rhomboid flap according to the present invention. The bottom row of images are of a hand drawn rhomboid flap. The flaps were on contralateral sides of the same cadaveric head.

In FIG. 7, a table of percent deviation in limb length and angle of projected and hand drawn rhomboid flaps is shown. The hand drawn flap had a 50% deviation in each angle and a 20% deviation in the length of one limb, from a theoretical flap angle and length when compared to published theoretical parameters. The projected flap had 0% deviation in each angle and limb measured.

Upon completion of the experiment, the surgeon was given a post-experimental survey (FIG. 8). A score of 5 correlates to strongly agree and a score of 1 correlates to strongly disagree. The average score was 4.91.

It should be understood, of course, that the foregoing relates to exemplary embodiments of the invention and that modifications may be made without departing from the spirit and scope of the invention as set forth in the following claims.

We claim:

1. A method of creating a skin flap, comprising:
selecting a skin flap configuration from a database of pre-existing skin flap configurations; and
projecting the selected skin flap configuration onto an area of skin.

2. The method of claim 1, further comprising:
modifying the selected skin flap configuration.

3. The method of claim 1, further comprising:
projecting a thermal image of the area of skin onto the area of skin.

4. The method of claim 1 further comprising:
overlaying a 3D picture of the area of skin onto the area of skin.

5. The method of claim 1, further comprising:
storing, in the database, a thermal image of blood supply of the area of skin.

6. The method of claim 1, further comprising:
storing, in the database, 3D images of skin surface topography and tissue volume of the area of skin.

7. The method of claim 1, further comprising:
imaging, of the area of skin, that includes at least one of color, texture, hair content, elasticity, collagen content, elastin content, and water content.

8. The method of claim 1, wherein selecting the skin flap configuration is selected by a user.

9. The method of claim 1, wherein selecting the skin flap configuration is selected by a computer.

10. A system of creating a skin flap, comprising:
a database of pre-existing skin flap configurations;
a processor in communication with the database; and
a projector in communication with the database and the processor;
wherein the projector is configured to project a selected, pre-existing skin flap configuration onto an area of skin.

11. The system of claim 10, further comprising:
a thermal imaging camera in communication with the database and the processor.

12. The system of claim 10, further comprising:
a 2D camera in communication with the database and the processor.

13. The system of claim 10, further comprising:
a 3D camera in communication with the database and the processor.

14. The system of claim 10, further comprising:
an imaging modality, in communication with the database and the processor, and configured to image the area of skin for at least one of color, texture, hair content, elasticity, collagen content, elastin content, and water content.

15. The system of claim 10, further comprising:
another database of at least one of skin surface topography and tissue volume of the area of skin.

16. The system of claim 10, further comprising:
another database of at least one of color, texture, hair content, elasticity, collagen content, elastin content, and water content of skin.

17. A non-transitory computer readable medium with computer executable instructions stored thereon, executed by a processor, to perform a method of creating a skin flap, the method comprising:
3D scanning to assess at least one of skin surface topography and tissue volume of a reconstruction area of skin;
using other imaging modalities to analyze and determine a skin characteristic of at least one of: color, texture, hair content, elasticity, collagen content, elastin content, and water content;
using at least one of the skin surface topography, the tissue volume, and the skin characteristic to select a skin flap and area to place the skin flap on the reconstruction area of skin; and
changing the selected skin flap to increase a fit of the selected skin flap to a patient.

18. The method of claim 17, wherein the 3D scanning includes thermally imaging the reconstruction area of skin.

19. The method of claim 17, further comprising projecting the selected skin flap on the reconstruction area.

20. The method of claim 19, wherein changing the selected skin flap occurs after projecting the selected skin flap.
